(19) United States
(12) Patent Application Publication
(10) Pub. No.: US 2020/0216800 A1
(43) Pub. Date: Jul. 9, 2020

(54) CELL CULTURE MEDIUM FOR PRODUCING TARGET MATERIAL AT HIGH EFFICIENCY BY USING MAMMALIAN CELLS, CELL CULTURING METHOD USING SAME, AND METHOD OF PRODUCING TARGET MATERIAL

(71) Applicant: Hong Woo PARK, (US)

(72) Inventors: Hong Woo PARK, Seoul (KR); Bong Gyun KIM, Gyeonggi-do (KR)

(73) Assignee: Hong Woo PARK, Seoul (KR)

(21) Appl. No.: 16/822,530

(22) Filed: Mar. 18, 2020

Related U.S. Application Data
(62) Division of application No. 15/560,032, filed on Sep. 20, 2017.

(30) Foreign Application Priority Data
Mar. 23, 2015 (KR) 10-2015-0040175

(51) Int. Cl. C12N 5/00; C12P 21/02; C12P 21/00; C07K 16/00

(52) U.S. Cl. CPC C12N 5/005; C12P 21/02; C12N 2500/22; C07K 16/00; C07K 2317/14; C12P 21/00

(57) ABSTRACT

The present invention relates to a cell culture medium using mammalian cells for producing a target material at high efficiency in the mammalian cells, a cell culturing method using same, and a method of producing the target material and, more particularly, to a cell culture medium including Zn ions at a concentration of 30μM or more in a culture, a salt thereof, or a chelate compound, a cell culturing method using same, and a method of producing a target material. According to the present invention, a cell culture medium for producing recombinant protein by using mammalian cells can be provided, which can achieve excellent effects in increasing antibody production, without showing any cell growth inhibiting effects.

### TECHNICAL FIELD

The present invention relates to a cell culture medium for the production of a target substance in mammalian cells with high efficiency, a method for culturing cells using the cell culture medium, and a method for the production of a target substance using the cell culture medium.

### BACKGROUND ART

Chinese hamster ovary (CHO) cells are widely used to produce recombinant protein therapeutics in the biopharmaceutical field due to their very rapid growth, high stability, and ability to effectively express transgenes. In this case, chemically defined media (CDM) characterized by high cell density and high production rate are preferred for the mass production of monoclonal antibodies because of economic reasons and safety problems associated with transmissible spongiform encephalopathy and other contamination sources. However, common chemically defined media suitable for all recombinant CHO (rCHO) cell lines have never been reported before because various cell lines are auxotrophic for different nutrients.

Many studies have been reported to increase the production of monoclonal antibodies in suspension cultures of rCHO cells. For example, studies aimed at increasing the production of recombinant proteins by adding butyrate, dimethyl sulfoxide, and pentanoic acid to a rCHO cell culture medium have been reported (Mimura Y, Lund J, Church S, Dong S, Li J, Goodall M, Jefferis R (2001) Butyrate increases production of human chimeric IgG in CHO-K1 cells whilst maintaining function and glycoform profile. J Immunol methods 247:205-216; Liu CH, Chu IM, Hwang SM (2001) Enhanced expression of various exogenous genes in recombinant Chinese hamster ovary cells in presence of dimethyl sulfoxide. Biotechnol Lett 23:1641-1645). However, the toxic effects of the compounds on rCHO cells limit the use of the compounds at high concentrations for the purpose of maximizing the production of proteins despite the positive effects observed in the production of recombinant proteins.

Hamilton's and Ham's groups reported the importance of adding trace elements for optimal growth of CHO cells in protein-free media (PFM), such as MCDB 301 and MCDB 302 (Hamilton WG, Ham RG (1977) Clonal growth of Chinese hamster cell lines in protein-free media. In Vitro 13:537-547). Further, the addition of trace metal elements to concentrate supplements in CHO cell-based fed-batch suspension culture extends the life of cells, leading to a significant increase in the production of monoclonal antibodies (Huang Y M, Hu W, Rustandi E, chang K, Yusuf-Makagiansar H, Ryll T (2010) Maximizing productivity of CHO cell-based fed-batch culture using chemically defined media conditions and typical manufacturing equipment. Biotechnol Prog 26:1400-1410). However, little is known about the composition of metal ions in the concentrate supplements.

On the other hand, zinc (Zn) ion is known as a coenzyme that regulate at least 300 biological functions involved in DNA synthesis, protein synthesis, cell division, cell proliferation, apoptosis, energy production, etc. Many papers concerning the relevance of Zn ion to cell culture have been published for the last two decades. The largest portion (˜24%) of these papers is associated with the antioxidative activity of Zn ion and the second largest portion is associated with insulin-like effects of Zn ion or apoptosis inhibition by Zn ion. Most previous studies associated with Zn ion have focused on the role of Zn ion in maintaining the structure and function of the cell membrane, antioxidative activities (protection of sulfhydryl groups, inhibition of hydroxyl radical production, and induction of metallothionein as an antioxidant protein), anti-inflammatory and immune response regulation, apoptosis inhibition (promotion of cell division through the MAP kinase pathway, inhibition of caspase-3 activity, and increase of Bcl-2/Bax ratio), increase of mRNA stability, insulin replacement effect (hybridoma (CRL1606), myeloma (NSO), CHO cell (CHO-K1)), and inhibition of ribonuclease activity, etc. However, no research has been conducted on the effect of Zn ions in culture media for the production of recombinant proteins by suspension culture of mammalian cells, including CHO cells.

### DETAILED DESCRIPTION OF THE INVENTION

### Problems to be Solved by the Invention

Therefore, the present invention has been made in view of the problems of the prior art and is directed to providing a cell culture medium for the production of the largest possible amount of a target substance in mammalian cells, including CHO cells, without inducing cytotoxicity, a method for culturing cells using the cell culture medium, and a method for the production of a target substance using the cell culture medium.

### Means for Solving the Problems

In one aspect, the present invention provides a cell culture medium for the production of a target substance in mammalian cells, the cell culture medium including zinc (Zn) ions, a Zn salt or a Zn chelate compound at a concentration of 30 μM or more.

According to one embodiment of the present invention, the mammalian cells may be selected from the group consisting of Chinese hamster ovary (CHO) cells, baby hamster kidney (BHK) cells, human embryonic kidney (HEK) cells, murine myeloma (NSO or SP2/0) cells, human retina-derived (PerC6) cells, and combinations thereof.

According to a further embodiment of the present invention, the target substance may be selected from the group consisting of: monoclonal antibodies, recombinant antibodies, and immunoglobulins containing fragments of the antibodies; fusion proteins in which proteins or peptides are fused to constant domains (Fc) of antibodies; hormones; cytokines; enzymes; and combinations thereof.

According to another embodiment of the present invention, the cell culture medium may be a protein-free medium or a chemically defined medium.

According to another embodiment of the present invention, the Zn salt may be selected from the group consisting of ZnSO₄, ZnSO₃, Zn(NO₃)₂, Zn(H₂PO₄)₂, Zn₃(PO₄)₂, Zn(NO₃)₂, (C₆H₆O₇)₂Zn₃, Zn₃BO₆, ZnBr₂, ZnF₂, ZnCl₂, ZnI₂, (C₂H₃O₂)₂Zn, [ZnCO₃]₂. [Zn(OH)₂]₃, Zn(ClO₄)₂, ZnMoO₄, ZnTiO₃, ZnSeO₃, Zn(CN)₂, ZnSiF₆. 6H₂O, (C₄H₅O₂)₂Zn, ZnC₂O₄, Zn(BF₄)₂, and (C₇H₇O₃S)₂Zn, anhydrides and hydrates of the salts, and combinations thereof.

According to another embodiment of the present invention, the Zn chelate compound may be a chelate compound of Zn and one or more compounds selected from the group consisting of ethylenediaminetetraacetic acid (EDTA), ethylene glycol-bis(β-aminoethyl ether)-N, N, N′, N′-tetraacetic acid (EGTA), desferrioxamine mesylate, diethylenetriam inepentaacetic acid (DPTA), trans-1,2-diaminocyclohexane-N, N, N′, N′-tetraacetic acid (CDTA), N, N,N′, N′-tetram ethylethylened iamine (TMEDA), phthalocyanine, pyrithione, meso-tetraphenylporphyrin, 8-hydroxyquinolate, bis(hexamethyldisilazide), and di[bis(trifluoromethylsulfonyl)imide].

In a further aspect, the present invention provides a method for culturing cells using the cell culture medium.

According to one embodiment of the present invention, the Zn ions, the Zn salt or the Zn chelate compound may be added at a concentration of 30 μM or more to the cell culture medium before cell culture.

According to a further embodiment of the present invention, the Zn ions, the Zn salt or the Zn chelate compound may be added at a concentration of 30 μM or more to the cell culture medium during cell culture.

According to another embodiment of the present invention, the Zn ions, the Zn salt or the Zn chelate compound may be added intermittently to the cell culture medium during cell culture such that the final concentration is 30 μM or more.

According to another embodiment of the present invention, the Zn ions, the Zn salt or the Zn chelate compound may be added continuously to the cell culture medium during cell culture such that the final concentration is 30 μM or more.

In another aspect, the present invention provides a method for the production of a target substance using the cell culture medium, the method including culturing cells in the cell culture medium, allowing the cells to express a target substance, and separating the target substance from the cells.

### Effects Of The Invention

The cell culture medium and the cell culture method of the present invention can be used to produce antibody as a target substance in mammalian cells with greatly improved productivity without inhibiting the cell growth.

### BRIEF DESCRIPTION OF THE DRAWINGS

FIG. 1 shows changes in the maximum density of rCHO-1 cells and the maximum production rate of antibody in HY-PFM when trace elements were added at various concentrations (n=3).

FIG. 2 shows changes in the maximum density of rCHO-1 cells and the maximum production rate of antibody in HY-PFM when trace elements were individually added at concentrations 20-fold higher than those used in a control group (n=3).

FIG. 3 shows changes in the maximum density of rCHO-1 cells and the maximum production rate of antibody in HY-PFM when Zn was added at various concentrations (n=3).

FIG. 4 shows changes in the maximum density of rCHO-1 cells and the maximum production rate of antibody in HY-CDM when Zn was added at various concentrations (n=2).

FIG. 5 shows changes in the maximum density of rCHO-2 cells and the maximum production rate of antibody in HY-CDM when Zn was added at various concentrations (n=2).

FIGS. 6 and 7 show changes in the maximum density of rCHO-1 cells (FIG. 6) and the maximum level of antibody (FIG. 7) in four commercial CDM containing Zn ions at an enhanced concentration of 60 μM.

FIGS. 8 and 9 show the growth rates of rCHO-1 cells and the production rates of antibody during suspension culture of the cells in HY-PFM (FIG. 8) and HY-CDM (FIG. 9) (n=2).

### BEST MODE FOR CARRYING OUT THE INVENTION

The definitions of the terms used herein are as follows.

The term “medium” refers to a nutritive composition that assists in sustaining, propagating, and/or undifferentiating cells. The term “chemically defined medium (CDM)” as used herein refers to a medium in which all components can be described by their chemical formulae and are present in known concentrations. The term “protein-free medium (PFM)” as used herein refers to a medium that does not substantially include polypeptides but includes some unidentified oligopeptides derived from animal or vegetable sources.

The term “cells” refers to a cell population. The cells may be wild-type or recombinant. The term “cell culture” or “cell culture technique” or “cell culture process” refers to a method and conditions suitable for the survival and/or growth and/or undifferentiation of the cells.

The term “target substance” refers to any recombinant protein, cell, virus or genome that may be useful for research, diagnostic or therapeutic purposes. The target protein may include a mammalian protein or non-mammalian protein and may optionally include a receptor or a ligand. Exemplary target proteins include, but are not limited to: molecules, such as renin; growth hormones, including human growth hormones and bovine growth hormones; growth hormone releasing factors; parathyroid hormones; thyroid stimulating hormones; lipoproteins; alpha-1-antitrypsin; insulin A-chain: insulin B-chain; proinsulin; follicle stimulating hormones; calcitonin; luteinizing hormone; glucagon; clotting factors, such as factor VIIIC, factor IX, tissue factor, and von Willebrands factor; anti-clotting factors, such as Protein C; atrial natriuretic factor; lung surfactants; plasminogen activators, such as urokinase or human urine or tissue-type plasminogen activator (t-PA); bombesin; thrombin; hemopoietic growth factors; members of the TNF and TNF receptor (TNFR) family, such as tumor necrosis factor-alpha and -beta; CD40 ligand, Apo-2 ligand/TRAIL, DR4, DR5, DcR1, DcR2, DcR3, OPG, and Fas ligand; enkephalinase; RANTES (regulated on activation, normally T-cell expressed and secreted); human macrophage inflammatory protein (MIP-1-alpha); serum albumins, such as human serum albumin; Mullerian-inhibiting substance; relaxin A-chain; relaxin B-chain; prorelaxin; mouse gonadotropin-associated peptide; microbial proteins, such as beta-lactamase; DNase; IgE; cytotoxic T-lymphocyte-associated antigens (CTLAs), such as CTLA-4; inhibin; activin; vascular endothelial growth factor (VEGF); receptors for hormones or growth factors; protein A or D; rheumatoid factors; neurotrophic factors, such as bone-derived neurotrophic factor (BDNF), neurotrophin-3, −4, −5 or −6 (NT-3, NT-4, NT-5 or NT-6) or nerve growth factors, such as NGF-β; platelet-derived growth factor (PDGF); fibroblast growth factors, such as aFGF and bFGF; epidermal growth factor (EGF); transforming growth factors (TGFs), such as TGF-alpha and TGF-beta, including TGF-1, TGF-2, TGF-P3, TGF-P4 or TGF-P5; insulin-like growth factor-I and -II (IGF-I and IGF-II); des(1-3)-IGF-I (brain IGF-I), insulin-like growth factor binding proteins; CD proteins, such as CD-3, CD-4, CD-8, CD-19, and CD20; erythropoietin; osteoinductive factors; immunotoxins; bone morphogenetic proteins (BMPs); interferons, such as interferon-alpha, -beta, and -gamma; colony stimulating factors (CSFs), e.g., M-CSF, GM-CSF, and G-CSF; thrombopoietin (TPO); interleukins (ILs), e.g., IL-1 to IL-10; superoxide dismutase; T-cell receptors; surface membrane proteins; decay accelerating factor; viral antigens, such as portions of the AIDS envelope and gpl20; transport proteins; homing receptors; addressins; regulatory proteins; integrins, such as CD11a, CD11b, CD11c, CD18, ICAM, VLA-4, and VCAM; tumor-associated antigens, such as HER2, HER3 or HER4 receptor; variants and/or fragments of any of the above-listed polypeptides; antibodies against various protein antigens like CD proteins such as CD3, CD4, CD8, CD19, CD20, and CD34; members of the ErbB receptor family, such as the EGF receptor, HER2, HER3 or HER4 receptor; cell adhesion molecules, such as LFA-I, Mac1, p150, 95, VLA-4, ICAM-I, VCAM and αv/β3 integrin including either α or β subunits thereof (e.g., anti-CD11a, anti-CD18 or anti-CD11b antibodies); growth factors, such as VEGF; IgE; blood group antigens; flk2/flt3 receptor; obesity (OB) receptor; mpl receptor; CTLA-4; protein C; Apo-2L receptors, such as Apo-2 (DR5), DR4, DcR1, DcR2, and DcR3; variants and/or fragments of the above-identified antibodies; and fusion proteins, for example, fusion proteins of proteins, such as tumor necrosis factor receptor (TNFR), CTLA-4, vascular endothelial growth factor receptor-1 (VEGFR-1), vascular endothelial growth factor receptor-2 (VEGFR-2), thrombopoietin-binding peptide and lymphocyte function-associated antigen 3 (LFA-3), and Fc fragments of human immunoglobulin G-1.

It should be understood that the terms and words used in the specification and the claims are not to be construed as having common and dictionary meanings but are construed as having meanings and concepts corresponding to the technical spirit of the present invention in view of the principle that the inventor can define properly the concept of the terms and words in order to describe his/her invention with the best method. Therefore, embodiments described in the specification and constructions illustrated in the drawings are provided for illustrative purposes only and are not intended to represent all the technical spirit of the present invention. Therefore, it should be understood that various equivalents and modifications can be made to these embodiments and constructions at the time of filing the present application.

The present invention will now be described in more detail with reference to the accompanying drawings and the following examples.

The present inventors have analyzed in all aspects the influences of various components on the growth of mammalian cells and the production of a target substance in culture media, and as a result, found that the presence of Zn ions at a predetermined concentration in the media can maximize the production of the target substance without inducing toxicity to the cell growth. The present invention has been accomplished based on this finding.

Zn ion is known as a coenzyme that regulate at least 300 biological functions involved in DNA synthesis, protein synthesis, cell division, cell proliferation, apoptosis, energy production, etc. However, most of the currently available cell culture media are free of Zn ion as an essential component. Even if present, the concentration of Zn ions is at most 3 μM. For example, the contents of Zn ions in MCDB, Ham's F-10, and Ham's F-12 are as low as 0.1-3.0 μM, 0.1 μM, and 3 μM, respectively. Zn ions may be added as medium supplements. In this case as well, the content of Zn ions is limited to 10 μM or less. For serum and protein-free media, Zn ions are added at concentrations of 0.1-3.1 μM and 3.02-9.10 μM to basal media, respectively. For most commercial chemically defined media, Zn ions are added at concentrations of 10 μM or less (for example, Power CHO2 (9.2 μM), HyCell CHO (11.9 μM), CDM4CHO (7.1 μM), Excell CD CHO (<1.5 μM), ProCHO5 (8.3 μM), CD OptiCHO (6.4 μM)).

As can be seen from the Examples section that follows, when cells were cultured in protein-free media and chemically defined media in the presence of various trace elements at controlled concentrations, Zn ions caused significant changes in the maximum density of the cells and the maximum level of a target substance but the trace elements other than Zn caused no significant changes in the maximum density of the cells and the maximum level of the target substance despite their varying concentrations or contents. It was also found that the addition of Zn ions at various concentrations, particularly at a concentration of 30 μM or more, to media leads to a considerable improvement in specific antibody production rate, and particularly the presence of Zn ions at concentrations of 30 μM to 90 μM in media is effective in producing a target substance without inhibiting the growth of cells.

Thus, the present invention provides a cell culture medium for the production of a target substance in mammalian cells, the cell culture medium including zinc (Zn) ions, a Zn salt or a Zn chelate compound at a concentration of 30 μM or more.

The cell culture medium of the present invention is suitable for the culture of mammalian cells. Chinese hamster ovary cells are preferred as mammalian cells taking into consideration their very rapid growth, high stability, and ability to effectively express transgenes. Other examples of suitable mammalian cells include, but are not necessarily limited to, baby hamster kidney (BHK) cells, human embryonic kidney (HEK) cells, murine myeloma (NS0 or SP2/0) cells, and human retina-derived (PerC6) cells.

The cell culture medium of the present invention is also designed to produce a target substance by cell culture. Final target substances produced using the cell culture medium of the present invention include the substances listed in the definition of the terms but are not limited thereto. Particularly, the target substance may be selected from the group consisting of: monoclonal antibodies, recombinant antibodies, and immunoglobulins containing fragments of the antibodies; fusion proteins in which proteins or peptides are fused to constant domains (Fc) of antibodies; hormones; cytokines; enzymes; and combinations thereof.

It was found that when Zn ions are enhanced to a predetermined concentration, a large amount of a target substance is effectively produced in chemically defined media as well as in protein-free media.

Various forms of Zn may be added to the medium so long as Zn ions are present at the concentration defined above. Zn may be in the form of a salt or chelate compound. All pharmaceutically acceptable Zn salts are possible, for example: ZnSO₄, ZnSO₃, Zn(NO₃)₂, Zn(H₂PO₄)₂, Zn₃(PO₄)₂, Zn(NO₃)₂, (C₆H₅O₇)₂Zn₃, Zn₃BO₆, ZnBr₂, ZnF₂, ZnCl₂, ZnI₂, (C₂H₃O₂)₂Zn, [ZnCO₃]₂. [Zn(OH)₂]₃, Zn(ClO₄)₂, ZnMoO₄, ZnTiO₃, ZnSeO₃, Zn(CN)₂, ZnSiF₆. 6H₂O, (C₄H₅O₂)₂Zn, ZnC₂O₄, Zn(BF₄)₂, and (C₇H₇O₃S)₂Zn, and anhydrides and hydrates of the salts. Suitable Zn chelate compounds may be chelate compounds of Zn and one or more compounds selected from the group consisting of ethylenediaminetetraacetic acid (EDTA), ethylene glycol-bis(β-aminoethyl ether)-N, N, N′, N′-tetraacetic acid (EGTA), desferrioxamine mesylate, diethylenetriam inepentaacetic acid (DPTA), trans-1,2-diaminocyclohexane-N, N, N′, N′-tetraacetic acid (CDTA), N, N,N′, N′-tetram ethylethylenediamine (TMEDA), phthalocyanine, pyrithione, meso-tetraphenylporphyrin, 8-hydroxyquinolate, bis(hexamethyldisilazide), and di[bis(trifluoromethylsulfonyl)imide].

The present invention also provides a method for culturing cells using the cell culture medium. The cell culture medium of the present invention is suitable for batch culture, fed-batch culture, and continuous culture. According to the cell culture method of the present invention, cells can be cultured in various ways so long as the concentration of the Zn ions, the Zn salt or the Zn chelate compound in the cell culture medium is 30 μM or more, as described above.

For example, the Zn ions, the Zn salt or the Zn chelate compound may be added at a concentration of 30 μM or more to the medium before or during cell culture. The Zn ions, the Zn salt or the Zn chelate compound may be added intermittently or continuously at a concentration of 30 μM or more to the medium during cell culture, Particularly, the culture method of the present invention can minimize the inhibition of cell growth caused by the presence of Zn ions at a high concentration, for example, at a concentration of 90 μM or more. Thus, the culture method of the present invention is advantageously fed-batch culture.

The present invention also provides a method for the production of a target substance using the cell culture medium. Specifically, the method includes culturing cells in the cell culture medium, allowing the cells to express a target substance, and separating the target substance from the cells.

In the step of culturing cells in the cell culture medium, it is very important to choose a suitable culture process for cell growth and establish suitable culture conditions, including culture temperature, culture medium composition, and when or how much to add the medium supplements, such that high productivity per unit medium is achieved. High productivity of the target substance can be achieved by the use of a cell line suitable for stable production of the target substance to increase the density of cells and optimization of the culture period. To this end, the choice of the cell culture medium and the medium composition is the most important factor. As described previously, the present inventors have analyzed in all aspects the influences of various components on the growth of mammalian cells and the production of a target substance in culture media, and as a result, found that the presence of Zn ions, a Zn salt or a Zn chelate compound at a concentration of 30 μM or more in the media can maximize the production of the target substance without inducing toxicity to the cell growth. The method of the present invention enables the production of a target substance with improved productivity compared to prior art methods.

The present invention will be explained in more detail with reference to the following examples. These examples are provided to assist in understanding the invention and are not intended to limit the scope of the invention.

Cell Line

Two recombinant CHO DG44 cell lines (hereinafter referred to as “rCHo-1” and “rCHO-2”) were used for monoclonal antibody (mAB) production. Each cell line was passaged 8 times or more and suspension adapted in a protein-free medium (HY-PFM, in-house) or chemically defined medium (HY-CDM, in-house). The suspension-adapted cell line was used to evaluate how the concentrations of trace elements, the selection of an effective trace element, and the concentration of ZnSO₄.7H₂O (Zn) affected the growth of cells and the production of antibody in commercial chemically defined media.

Cell Suspension Culture

The cell line was inoculated at a density of 4×10⁵ cells/ml and cultured in an Erlenmeyer flask with a 30 ml/125 ml working volume on an orbital shaker at 120 rpm under humidified conditions at 37° C. and 5% CO₂. Before inoculation, the cells were centrifuged at 1,000 rpm (×162 g) for 5 min, the supernatant was discarded, and the remaining cells were dispersed into single cells in the heated medium. The cells were passaged three times to minimize the influence of the remaining medium under the experimental conditions. The subcultured cells were evaluated.

Density of Live Cells

The density of live cells was analyzed using a hemocytometer (Neubauer improved bright-line, Marienfeld, Germany), an inverted microscope (CK30, Olympus, Japan), and trypan blue dye exclusion.

Antibody Level Analysis

4, 6, 8, 10, and 12 days after cells were inoculated under the experimental conditions, cell culture fluids were sampled, centrifuged at 1,000 rpm (×162 g) for 5 min, and stored at −20° C. before antibody analysis. Antibody levels were measured by sandwich enzyme linked immunosorbent assay (ELISA). For the detection of mAB by ELISA, anti-human IgG (Fab specific) (I5260, Sigma, St. Louis, Mo., USA) was used as a primary antibody and human IgG (Fc specific)-horseradish peroxidase (A0170, Sigma) was used as a chromogenic antibody. 1% (w/v) bovine serum albumin/phosphate buffered saline was used for blocking, 3,3′,5,5′-tetramethylbenzidine (TMB) solution (KPL, Gaithersburg, Md., USA) was used as a chromogenic reagent. The chromogenic reaction was stopped with 2 M H₂SO₄. For antibody level analysis, absorbance values were measured at a wavelength of 450 nm using a kinetic microplate reader (Molecular Devices, Sunnyvale, Calif., USA).

### EXAMPLE 1

### Concentration Ranges of Trace Elements Effective in Improving the production of antibody in suspension culture of rCHO-1 cell line using HY-PFM

HY-PFM containing 0.01 μM CuSO₄.5H₂O (Cu), 3 μM ZnSO₄.7H₂O (Zn), 0.03 μM Na₂SeO₃ (Se), 0.01 μM NH₄VO₃ (V), 0.001 μM MnSO₄.H₂O (Mn), and 0.01 μM (NR₄)₆Mo₇O₂₄.4H₂O (Mo) as trace element sources was used as a control group. In order to evaluate the concentration ranges of the trace elements effective for antibody production, the concentration of the trace element mixture was increased to 1-40 times that in the control group. After cells were cultured, maximum antibody levels and maximum cell densities were measured.

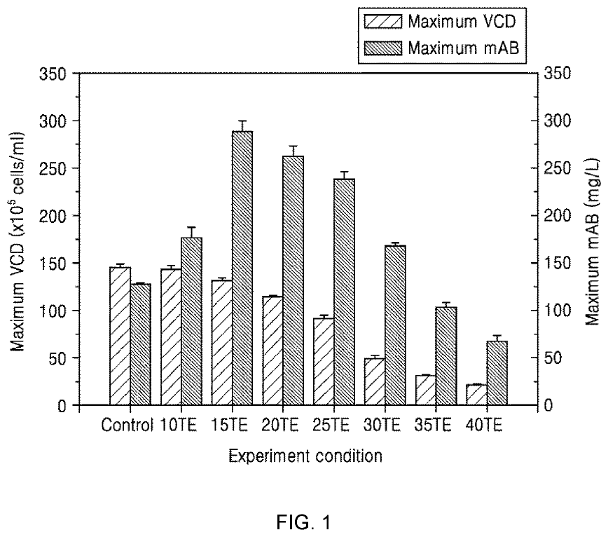

FIG. 1 shows changes in the maximum density of rCHO-1 cells and the maximum production rate of antibody in HY-PFM when the trace elements were added at various concentrations (n=3). Referring to FIG. 1, the addition of the trace elements in a 10-fold amount increased the maximum cell density to a level similar to that in the control group, and thereafter, the maximum cell density decreased with increasing amount of the trace elements. When the trace elements were added in 15-fold to 25-fold amounts, the maximum cell densities were 1.2×10⁷ cells/ml, which correspond to ≥˜80% of that in the control group, and the maximum antibody levels were ≥˜240 mg/L, which are 1.9 times higher than that in the control.

### EXAMPLE 2

### Selection of Component that Improves Antibody Production

In order to find a trace element that improves antibody production, rCHO-1 cell line was subjected to suspension culture in HY-PFM. The content of each of the trace elements Cu, Zn, Se, V, Mn, and Mo in HY-PFM was increased to 20-fold higher than that used in the control. Maximum cell densities and maximum antibody levels were compared and evaluated.

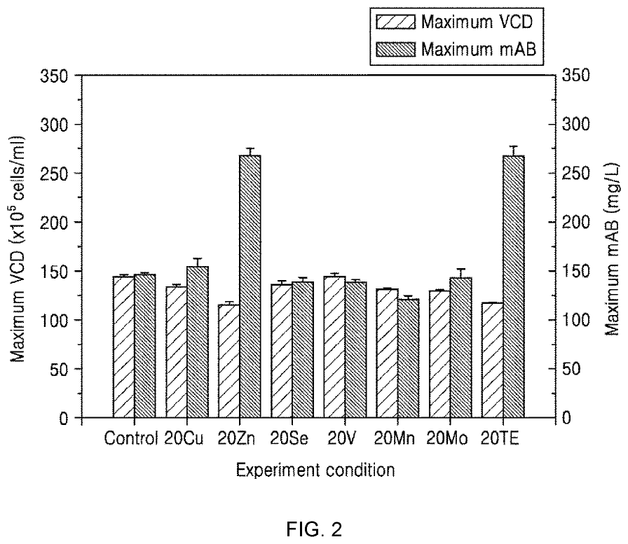

FIG. 2 shows changes in the maximum density of rCHO-1 cells and the maximum production rate of antibody in HY-PFM when the trace elements were individually added at concentrations 20-fold higher than those used in the control group (n=3). Referring to FIG. 2, the addition of Zn in a 20-fold amount increased the maximum cell density and the maximum antibody level to 1.1×10⁷ cells/ml and 260 mg/L, respectively, which are similar to those obtained when all trace elements were added in 20-fold amounts. The maximum cell densities and the maximum antibody levels obtained when the trace elements other than Zn were added in 20-fold amounts were similar to those in the control group. These results can lead to the conclusion that the addition of Zn at a high concentration effectively increases the antibody production.

### EXAMPLE 3

### Concentration of Zn Effective in Improving the Production of Antibody in Suspension Culture of rCHO-1 Cell Line using HY-PFM

In order to compare and evaluate the influences of the concentration of Zn on the maximum cell density and the maximum antibody production, the concentration of Zn in HY-PFM was changed in the range of 3-120 μM.

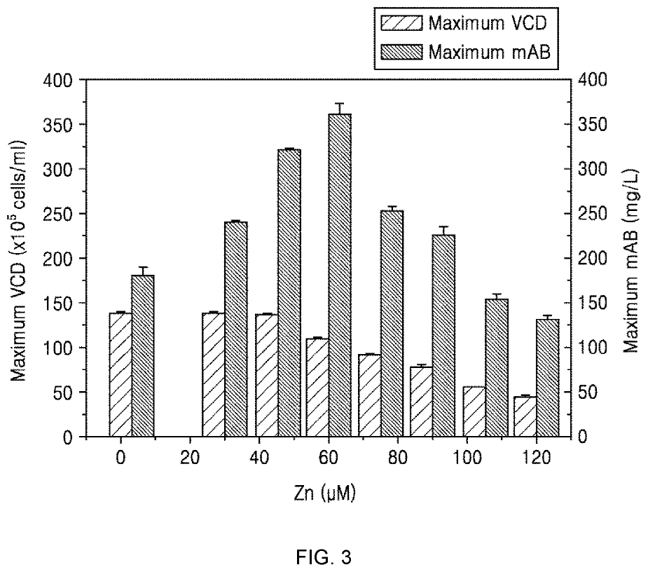

FIG. 3 shows changes in the maximum density of rCHO-1 cells and the maximum production rate of antibody in HY-PFM when Zn was added at various concentrations (n=3). Referring to FIG. 3, when Zn was added at concentrations of 45-60 μM, the antibody production reached a maximum of 360 mg/L, which corresponds to twice that in the control group. Cell densities obtained at Zn concentrations up to 45 μM were similar to that in the control group.

### EXAMPLE 4

### Concentrations of Zn Effective in Improving the Production of Antibody in Suspension Culture of rCHO-1 and rCHO-2 Cell Lines using HY-CDM

In order to exclude the influence of enzyme-derived hydrolysates as composite substances present in HY-PFM on antibody production, the experimental procedure of Example 3 was repeated except that HY-CDM composed of chemically defined components only were used.

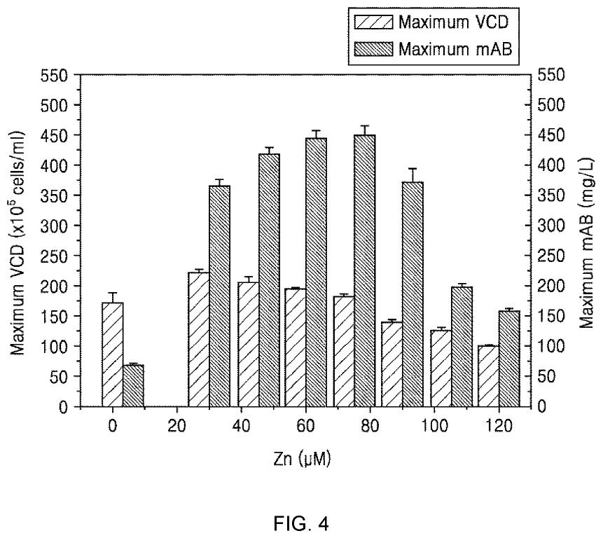

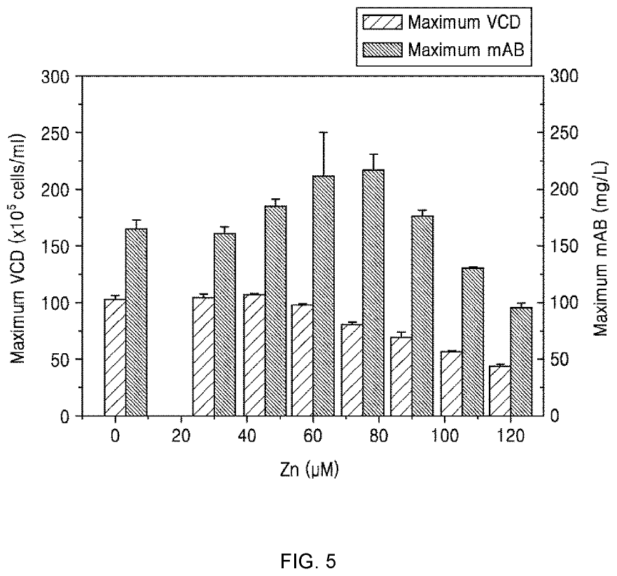

FIGS. 4 and 5 show changes in the maximum density of rCHO-1 cells (FIG. 4, n=2) and rCHO-2 cells (FIG. 5, n=2) and the maximum production rate of antibody in HY-CDM when Zn was added at various concentrations. Referring to FIGS. 4 and 5, the addition of Zn at concentrations around 60-75 μM increased the antibody production rates to 440 mg/L and 210 mg/L, which correspond to 6.6-fold (rCHO-1) and 1.3-fold (rCHO-2) compared to those in the control group. For the rCHO-2 cell line, the cell densities at Zn concentrations up to 45 μM were similar to that in the control group. In contrast, for the rCHO-2 cell line, the cell densities at Zn concentrations up to 45 μM were-1.2 times higher than that in the control group.

### EXAMPLE 5

### Improving Effect of Zn Addition on Antibody Productivity in Suspension Culture of rCHO-1 Cell Line using Commercial Chemically defined Media

In this example, the versatility of the effect of Zn ion (60 μM concentration) addition to improve antibody productivity was evaluated. To this end, four commercial chemically defined media listed in Table 1 were chosen and used to evaluate antibody productivity depending on the addition of Zn.

TABLE 1

| | | Zinc | |
|---|---|---|---|
| | Catalog No. | content | |
| Medium | (Manufacturer) | (μM)^{a} | Main features |
| PowerCHO-2 | BE12-771Q | 9 | Chemically defined medium |
| CD (CDM-1) | (Lonza) | | free of serum, animal- |
| | | | derived components, and |
| | | | hydrolysates and containing |
| | | | slight amount of recombinant |
| | | | human insulin |
| CDM4CHO | SH30557.02 | 7 | Chemically defined medium |
| (CDM-2) | (Hyclone) | | free of animal-derived |
| | | | components |
| Ex-CELL CD | 14360C | <1.5^{b} | Chemically defined medium |
| CHO (CDM-3) | (SAFC) | | free of animal-derived |
| | | | components and serum and |
| | | | supplemented with 0.1 mg/L |
| | | | recombinant protein |
| CD OptiCHO | 12681-011 | 6 | Chemically defined medium |
| (CDM-4) | (Gibco) | | free of serum, proteins, |
| | | | animal-derived components, |
| | | | hydrolysates, and unknown |
| | | | components |

^{a}the contents of Zn ions in the media were analyzed at the Korean Basic Science Institute (Seoul).
^{b}the contents of Zn ions in the media were analyzed with a limit of detection of 1.5 μM or less.

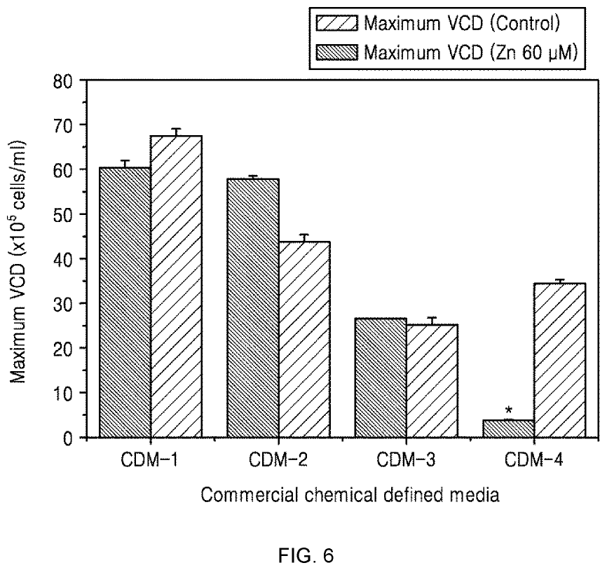

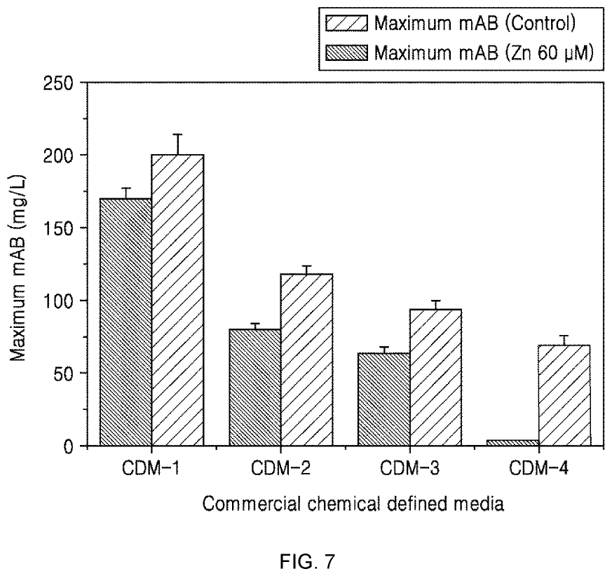

FIGS. 6 and 7 show changes in the maximum density of rCHO-1 cells (FIG. 6) and the maximum level of antibody (FIG. 7) in the four commercial CDM containing Zn ions at an enhanced concentration of 60 μM. Referring to FIGS. 6 and 7, the antibody productivities in CDM-1, 2, and 3 were 1.2-1.5 times higher than those in the control group. No inhibition of cell growth was observed in CDM-1, 2, and 3. In contrast, only continuous cell growth and antibody production was observed in CDM-4.

### EXAMPLE 6

### Analysis of Cell Growth and Antibody Production in Suspension Culture of rCHO-1 Cells with Zn at Enhanced Concentration of 60 μM

In this example, rCHO-1 cells were subjected to suspension culture in HY-PFM and HY-CDM containing Zn at an enhanced concentration (60 μM) and then specific antibody production rates (q_{mAB}) and cell longevities were analyzed as main factors in antibody production.

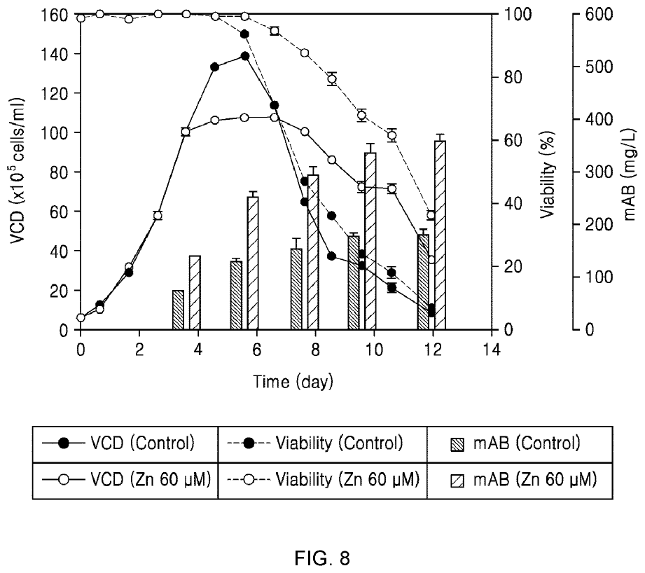

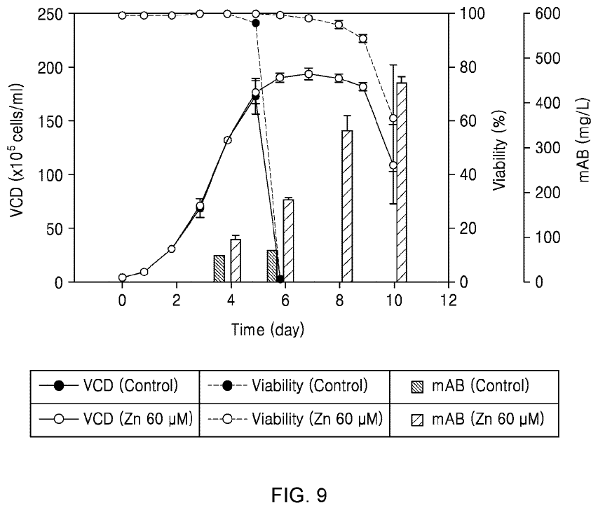

FIGS. 8 and 9 and Table 2 show the cell growth rates and the antibody production rates during suspension culture of the rCHO-1 cells in HY-PFM (FIG. 8) and HY-CDM (FIG. 9) (n=2). Referring to FIGS. 8 and 9 and Table 2, the q_{mAB} g values were 9.4 and 5.1 pcd at the initial stage of cell culture (culture period: 0-4 days), which correspond to at least 1.7 times those in the control group. The q_{mAB} g values at the late stage (culture period: 4-8 days) in both media were 2.1 times higher than those in the control group. The cell longevities representing a cell viability of 80% in HY-PFM and HY-CDM were 1.4 and 1.8 times higher than those in the control group, respectively. The live cell densities were 9×10⁶ cells/ml, which were maintained during cell culture for at least 9 days. These effects of Zn addition led to a ≥2-fold increase in antibody production compared to those in the control group.

TABLE 2

| | | | | Cell | |
|---|---|---|---|---|---|
| | | | | longevity | |
| Zn | q_{mAB} (ped, | q_{mAB} | | (day/80% | Culture |
| (μM) | 0-4 days) | (ped, 4-8 days) | μ (day⁻¹) | viability) | medium |
| 3 | 4.99 ± 0.13 | 1.75 ± 0.01 | 0.87 ± 0.01 | 6.2 ± 0.0 | PFM |
| 60 | 9.35 ± 0.11 | 3.74 ± 0.04 | 0.90 ± 0.02 | 8.5 ± 0.2 | |
| 3 | 3.07 ± 0.19 | 1.47 ± 0.10^{a} | 1.01 ± 0.04 | 5.1 ± 0.0 | CDM |
| 60 | 5.06 ± 0.07 | 3.30 ± 0.01 | 1.00 ± 0.00 | 9.3 ± 0.4 | |

^{a}q_{mAB} was calculated from the results obtained 4-6 days after cell culture

From the above results, it can be concluded that when the concentration of Zn ions in the medium composition of the present invention for suspension culture of recombinant CHO cell lines in both protein-free and chemically defined media is enhanced to 30 μM, the specific production rates of antibody are improved. Particularly, when the Zn ion concentration is enhanced to 30-60 μM, the medium composition of the present invention has no inhibitory effect on cell growth. When the Zn ion concentration is enhanced to 30-90 μM, the medium composition of the present invention is very effective in producing antibody by batch culture.

1.-12. (canceled)

13. A method for culturing cells using a cell culture medium for the production of a target substance in mammalian cells,
comprising culturing cells by suspension culture method in the cell culture medium containing zinc (Zn) ions, a Zn salt or a Zn chelate compound at a concentration of 30 μM or more,
wherein the mammalian cells are Chinese hamster ovary (CHO) cells, and
wherein the cell culture medium is a protein-free medium or a chemically defined medium for suspension culture.

14. The method according to claim 13, wherein the Zn ions, the Zn salt or the Zn chelate compound is added at a concentration of 30 μM or more to the cell culture medium before cell culture.

15. The method according to claim 13, wherein the Zn ions, the Zn salt or the Zn chelate compound is added at a concentration of 30 μM or more to the cell culture medium during cell culture.

16. The method according to claim 13, wherein the Zn ions, the Zn salt or the Zn chelate compound is added intermittently to the cell culture medium during cell culture such that the final concentration is 30 μM or more.

17. The method according to claim 13, wherein the Zn ions, the Zn salt or the Zn chelate compound is added continuously to the cell culture medium during cell culture such that the final concentration is 30 μM or more.

18. The method according to claim 13, wherein the target substance is selected from the group consisting of: monoclonal antibodies, recombinant antibodies, and immunoglobulins containing fragments of the antibodies; fusion proteins in which proteins or peptides are fused to constant domains (Fc) of antibodies; hormones; cytokines; enzymes; and combinations thereof.

19. The method according to claim 1, wherein the Zn salt is selected from the group consisting of ZnSO_{4,} ZnSO_{3,} Zn(NO₃)_{2,} Zn(H₂PO₄)_{2,} Zn₃(PO₄)_{2,} Zn(NO₃)_{2,}(C₆H₅O₇)₂Zn_{3,} Zn₃BO_{6,} ZnBr_{2,} ZnF_{2,} ZnCl_{2,} ZnI_{2,} (C₂H₃O₂)₂Zn, [ZnCO₃]₂. [Zn(OH)₂]₃, Zn(ClO₄)_{2,} ZnMoO_{4,} ZnTiO_{3,} ZnSeO_{3,} Zn(CN)_{2,} ZnSiF₆. 6H₂O, (C₄H₅O₂)₂Zn, ZnC₂O_{4,} Zn(BF₄)_{2,} and (C₇H₇O₃S)₂Zn, anhydrides and hydrates of the salts, and combinations thereof.

20. The method according to claim 13, wherein the Zn chelate compound is a chelate compound of Zn and one or more compounds selected from the group consisting of ethylenediaminetetraacetic acid (EDTA), ethylene glycol-bis(β-aminoethyl ether)-N, N, N′, N′-tetraacetic acid (EGTA), desferrioxamine mesylate, diethylenetriam inepentaacetic acid (DTPA), trans-1,2-diaminocyclohexane-N, N, N′, N′-tetraacetic acid (CDTA), N, N, N′, N′-tetram ethylethylenediamine (TMEDA), phthalocyanine, pyrithione, meso-tetraphenylporphyrin, 8-hydroxyquinolate, bis(hexamethyldisilazide), and di[bis(trifluoromethylsulfonyl)imide].
